(19) United States
(12) Patent Application Publication
(10) Pub. No.: US 2010/0121200 A1
(43) Pub. Date: May 13, 2010

(54) DIAGNOSTIC AND PROGNOSTIC ASSISTANCE DEVICE FOR PHYSIOPATHOLOGICAL TISSUE CHANGES

(75) Inventors: Odile Carvalho, Champigny Sur Marne (FR); Laurence Roy, Versailles (FR); Marc Benderitter, L'Hay-Les-Roses (FR)

Correspondence Address: PEARNE & GORDON LLP, 1801 EAST 9TH STREET, SUITE 1200, CLEVELAND, OH, 44114-3108, US

(73) Assignee: INSTITUT DE RADIOPROTECTION ET DE SURETE NUCLEAIRE, Fontenay-Aux-Roses (FR)

(21) Appl. No.: 12/596,025

(22) PCT Filed: Apr. 18, 2008
(86) PCT No.: PCT/EP2008/054764

§ 371 Date: Oct. 23, 2009

(30) Foreign Application Priority Data
Apr. 19, 2007 (FR) 07 54570

(51) Int. Cl. A61B 6/00

(52) U.S. Cl. 600/476

(57) ABSTRACT

Said device applies in particular to in vivo diagnostic and prognostic assistance for physiopathological changes to biological tissues. It comprises a coherent light source (13) for emitting coherent light along a first direction (X), for the purpose of illuminating a biological tissue (16) in first and second areas thereof, the first area being sound and the second area liable to include changes, the tissue thus illuminated generating a speckle phenomenon, means (14) for observing the speckle field in a second direction (Y) and for acquiring the speckle, and means (18) for varying the angle between the first and second directions, in order to observe the speckle field at different angles, so as to acquire information about the tissue at various depths in this tissue, support and absorption means (20, 28) to maintain a constant distance between the surface of the tissue and the observation and acquisition means and to absorb possible movements of the tissue, electronic means (22) for processing the speckle figures obtained by means of the observation and acquisition means, said processing being carried out in particular by a fractal method, in order to compare the first and second areas, and electronic means (24) for analysing the processing of the figures, by statistical methods, making it possible to validate the comparison made between the first and second areas.

### TECHNICAL FIELD

The present invention relates to a device for measuring, in vivo, properties of biological tissues for diagnostic and prognostic assistance for physiopathological changes to said tissues.

It especially applies to cutaneous tissular lesions and in particular to damage linked to irradiation.

### STATE OF THE PRIOR ART

Radiological burns result from a cascade of complex biological and molecular mechanisms that can lead to their non-repair and to the destruction of cutaneous tissue (see document [1] which, like the other documents cited hereafter, is detailed at the end of the present description).

The progressive instauration of a chronic inflammation, an angiogenesis defect, an abnormal remodelling of the extracellular matrix and a re-epithelisation defect is at the origin of radio-induced damage. The complexity of this tissular response results from radiosensitivity differences of each type of cell involved and their intercellular communications.

Cutaneous radiological burns are a syndrome whose clinical effects are known but are difficult to predict, whether in the short or in the long term. Indeed, unlike thermal burns, the visible consequences of such burns (erythema, oedema, necrosis, etc.) do not appear immediately after exposure to the irradiation source. A variable latency time depends in particular on the irradiation dose, the volume of the irradiated tissue, the irradiation source, the exposure time and the specific response of each individual. This biological latency time is also known as the clinically silent phase.

Knowledge of the dose and its biological effects is thus one of the determining factors for the diagnostic, the prognostic and the treatment of radiological burns (see document [2]). Thus, doses greater than 20-25 Gy lead to a necrosis of the irradiated tissues and generally necessitate the ablation of the irradiated tissues to prepare a skin graft. The quicker this surgical act is performed, the more favourable the prognostic. The medical management of radiological burns thus entirely depends on the quality of the diagnostic. At present, no device exists that makes it possible to assure a reliable diagnostic.

A radiological burn is a clinical situation that may be encountered within the context of accidental exposures to ionising radiation but also within the context of controlled radiotherapy exposures.

As regards irradiation accidents, unfortunately still frequent today, nearly 600 radiological accidents have been listed throughout the world since 1945. Among these, 78% correspond to localised irradiations and 22% to overall irradiations.

In certain cases, it is possible, by mathematical modelling, to establish a mapping of the irradiated area, but this requires a very precise knowledge of the nature and the localisation of the source, the volume of irradiated tissue and the exposure time, given that such information is not generally available in the case of an accident.

Only biopsy makes it possible to reveal an irradiated tissue and to evaluate the dose received: histological measurements on a skin biopsy make it possible to reveal the irradiation of the tissue, and bone biopsy makes it possible to quantify precisely the dose received by Electron Parametric Resonance (EPR). However, biopsy constitutes an invasive surgical act that surgeons are apprehensive about, since it is capable of aggravating the condition of the tissue already weakened by the irradiation.

When the dose is above 20-25 Gy, the irradiation causes serious cutaneous lesions and, even though the pathogeny of ionising radiation effects on cutaneous tissues is well documented, the medical response still remains extremely complex and delicate, particularly because the diagnostic remains difficult.

It is consequently vital to develop experimental protocols, which are non-invasive and can be used in vivo, for assisting the medical diagnosis of cutaneous irradiations.

As regards radiotherapy, nearly 30% of patients develop a cutaneous toxicity and 5% of patients unfortunately develop severe complications. Radiotherapy is based on the optimisation of the prescribed dose to destroy the tumour while preserving surrounding sound tissue included in the irradiation field. The risk of secondary complications linked to the exposure of sound tissues to ionising radiation is thus inevitable.

The severity of these lesions depends on several factors, such as the radiosensitivity of the tissue, the dose, the exposure frequency or even the pathological case histories of the patient. The acute toxicity of the radiotherapy vis-à-vis cutaneous tissues can bring about the stoppage of the treatment.

It is consequently vital to have a device that would make it possible to monitor the evolution of irradiated sound tissues in order to be able to diagnose and treat as quickly as possible an unfavourable evolution to protect the patient.

Several devices enable thermal burns to be diagnosed but they cannot be applied to the diagnostic of radiological burns since such tools do not provide any diagnostic element during the clinically silent phase of the radiological burn.

Among the devices that are used for clinical examinations in the case of thermal burns, infrared thermography techniques, vascular scintigraphy techniques or even Doppler laser techniques make it possible to reveal changes to the local blood flow.

In a context of radiological burns, infrared thermography and Doppler laser enable the irradiated area to be distinguished from the sound area during the first 48 hours after irradiation in mini pigs irradiated locally (40 Gy). After the forty eighth hour following irradiation, these techniques do not enable sound skin to be differentiated from irradiated skin.

Other techniques have been tested, particularly NMR imaging and X-ray tomography, which make it possible to reveal changes to the density and the hydration state of the tissues, characteristic of the oedema. These imaging techniques then make it possible to delimit an oedema, the density of which, closer to that of water, is lower than that of sound tissue. But, in a radiological burn context, such weighty and costly techniques do not make it possible to distinguish an irradiated tissue within a sound tissue during the clinically silent phase.

Table 1 summaries the different biophysical and biological techniques that are proposed as a function of the clinical evolution of the lesions (see document [3]). However, none of these techniques has ever made it possible to reveal an irradiated tissue compared to a sound tissue in the absence of visible clinical signs. Consequently, such techniques cannot be used clinically for diagnostic and prognostic assistance for cutaneous irradiation.

TABLE 1
Biophysical and biological methods that can be used as a function
of the type of tissular damage studied (see document [3])

| | Physiopathological | Biophysical or biological |
|---|---|---|
| Clinical change | changes | methods |
| Erythema | Increase in capillary | Thermography, vascular |
| Hyperhemia | permeability and blood | scintigraphy |
| Hyperthermia | flow | |
| Oedema | Plasma extravasation | X-ray scanning, NMR |
| | | imaging |
| Passive congestion | Reduction in blood flow | Thermography, |
| Thrombosis | | Vascular scintigraphy |
| Ischemia | Tissular anoxia | Vascular scintigraphy, |
| | | Cutaneous oxymetry |
| Necrosis | Cell destruction | Biochemical blood |
| | | labelling |

Some authors (see document [4]) have studied the depolarising properties of irradiated skin in pigs, in vitro, by the analysis of Müller matrices, representative of the polarising properties of the medium. The experiments carried out by these authors were conducted ex vivo, on biopsies of pig skins. The device is not currently applicable to living media on account of its physiological mobility (breathing, heart beat, etc.), which leads to problems of recycling of images. In addition, the device used is heavy and thus difficult to transport and quite costly.

### DESCRIPTION OF THE INVENTION

Thus, no non-invasive system that can be used in vivo is currently capable of assisting the diagnosis of the serious pathology constituted by cutaneous irradiation, even though it shows no clinical sign.

The present invention aims to overcome this drawback.

The technique, object of the invention, and its valorisation on a pre-clinical model constitute a progress for early diagnosis and prognosis and the health of the patient.

As will be seen, the device, object of the invention, enabling the acquisition and the processing of speckle figures particularly by a fractal approach, constitutes an advantageous device for in vivo diagnostic assistance for radiological burns and the prognostic of their evolution. The diagnostic and prognostic value of this device has been validated.

More precisely, the object of the present invention is a device for measuring, in vivo, properties of biological tissues, in particular for diagnostic and prognostic assistance for physiopathological changes, particularly tissular lesions and more specifically by irradiation, for the evaluation of cutaneous ageing, for the evaluation of the efficacy of cosmetological or dermatological products, said device being characterised in that it comprises:
a coherent light source for emitting a coherent light along a first direction, for the purpose of illuminating a biological tissue in first and second areas thereof, the first area being sound and the second area liable to include changes, the tissue thus illuminated generating a speckle phenomenon,
observation and acquisition means for observing the speckle field in a second direction and for acquiring the speckle,
means (mechanical or other) for varying the angle between the first and second directions, in order to observe the speckle field at different angles, so as to acquire information about the tissue at various depths in this tissue,
for the purpose of enabling the comparison of the first and second areas, support and absorption means (mechanical or other) to maintain a constant distance between the illumination point of the surface of the tissue and the observation and acquisition means and to absorb possible movements of the tissue, due to external factors, for example breathing,
electronic means for processing the speckle figures obtained by means of the observation and acquisition means, in order to compare the first and second areas, and
electronic means for analysing the processing of the figures, by statistical methods, making it possible to validate the comparison made between the first and second areas.

It should be noted that the means for varying the angle enable the acquisition of the speckle at several observation angles and thus the exploration of the tissue at several depths and, consequently, enable physiopathological changes of different layers of the tissue to be taken into account.

Furthermore, the above mentioned statistical methods are, for example, statistical tests or factorial analyses.

According to a preferred embodiment of the device, object of the invention, the means for varying the angle between the first and second directions are capable of varying said angle in the interval ranging substantially from 0° to 180° and making it possible to observe the speckle outside of the specular reflection, at several angles with respect to the direction of said specular reflection. This embodiment thus enables the preferential exploration of the different tissular layers, at various depths.

Preferably, the means for varying the angle between the first and second directions are capable of modifying the orientation of the first direction independently of that of the second direction and inversely.

According to a preferred embodiment of the device object of the invention,
the observation and acquisition means comprise photodetection means that capture the speckle and supply electrical signals representative of the corresponding speckle figures, and
the electronic processing means are capable of processing the electrical signals in the form of non-compressed images, and enabling the first and second areas to be compared.

Preferably, the photodetection means are capable of capturing the speckle with exposure times of at most 100 μs.

The photodetection means preferably comprise a camera.

The camera is preferably a camera without objective but may also be a camera with objective.

The camera is for example a CCD camera.

According to a preferred embodiment of the invention, the observation and acquisition means are provided to acquire at least 200 speckle figures per area illuminated.

The device, object of the invention, may further comprise optical means that are capable of controlling the polarisation of the coherent light emitted by the source and the polarisation of the light arriving on the observation and acquisition means, for the purpose of completing the selection of the speckle coming from the more or less deep layers of the tissue. These optical means comprise polarisers (linear, circular or elliptical) and/or half or quarter wave plates.

Preferably, the coherent light source is monochromatic.

Said source is, preferably, a laser.

Depending on the experimental conditions, particularly the type of camera used, the distance between the illumination point of the surface of the tissue and the camera is equal, preferably, to around 20 cm.

The processing of the speckle figures may be carried out by a conventional frequential method and/or by a fractal method.

According to a preferred embodiment of the invention, when the processing of the speckle figures is carried out by a fractal method, said processing comprises the extraction of stochastic parameters that are characteristic of speckle figures.

Preferably, the stochastic parameters comprise:
the Hurst coefficient,
the autosimilarity, and
the saturation of the variance.

### BRIEF DESCRIPTION OF DRAWINGS

The present invention will be better understood on reading the description of embodiment examples given hereafter solely by way indication and in no way limitative, by referring to the appended drawings in which:

FIG. 1 is a schematic view of a specific embodiment of the device, object of the invention,

FIGS. 2a and 2b show, in the case of the conventional frequential approach, the average speckle grain size for each measurement point and for each area, namely a sound area (dotted lines) and an irradiated area (solid lines), as regards the width of the grains dx (FIG. 2a) and the height of the grains dy (FIG. 2b), for a pig numbered P129, for an angle of incidence ψ of 20° of the light beam used for the formation of the speckle,

FIGS. 3a, 3b and 3c show, in the case of the fractal approach, fractal parameters, calculated along the horizontal dimension of the image, for each measurement point and for each area, namely a sound area (dotted lines) and an irradiated area (solid lines), as regards the saturation of the variance G (FIG. 3a), the autosimilarity S (FIG. 3b) and the Hurst coefficient H (FIG. 3c), for the pig mentioned above, with the same angle of incidence of the light beam,

FIG. 4 shows photographs of the irradiated area of the above mentioned pig skin at the measurement dates (40 Gy),

FIG. 5 is a representation of the scores on the different experimentation dates of the discriminating parameters for various angles, all pigs taken together,

FIG. 6 shows the increase in the thickness of the epidermis and that of the dermis, for an irradiated area compared to a sound area (in %) for four pigs,

FIG. 7 shows the evolution of the ratio 40 Gy/0 Gy for an observation angle of 20°, for three stochastic coefficients, namely the saturation of the variance G, the autosimilarity S and the Hurst coefficient H, as a function of the measurement dates, all measurement points taken together, for the pig numbered P129,

FIG. 8 shows the evolution of the Hurst coefficient H as a function of time, for each area, namely a sound area (dotted lines) and an irradiated area (solid lines), all measurement points taken together, for the angle of observation of 60° and the pig numbered P161.

FIG. 9 shows the power spectral density of a speckle figure (log-log scale),

FIG. 10 shows the normalised autocovariance function c_{I}(x,0), dx representing the full width at half maximum of the function, and

FIG. 11 is a log-log representation (arbitrary units) of the diffusion function of a speckle figure, obtained in the case of sound skin.

### DETAILED DESCRIPTION OF SPECIFIC EMBODIMENTS

We will firstly recall some aspects of the speckle phenomenon, followed by the state of the art of the applications of this phenomenon.

The speckle phenomenon is an interferential phenomenon, due to the interaction of coherent light with a diffusing medium. Such a medium has local fluctuations of density and thus refractive index. These local areas, randomly distributed in the medium, constitute partial wave diffusers. The random dephasing of these partial waves causes random interferences that induce a statistical intensity distribution. The intensity figure thus produced, of granular appearance, is known as “speckle”.

This phenomenon has for a long time been considered as a simple imaging noise. However it stems directly from light/matter interaction. Consequently, the parameters of the speckle (size of the grains, contrast, intensity, polarisation, etc.) can provide information on the properties of the medium and, in particular, on its optical properties, the main difficulty being quantitatively to acquire this information. This is the reason why, over the last few years, physicists have taken an interest in the exploitation of the speckle to characterise the medium that generates it.

Several applications have thus been developed: in stellar physics, in industry for measuring surface roughness or the deformation of objects, or instead in medical imaging, the field to which the present invention relates.

In this field, the measurement of the spatial and dynamic characteristics of the speckle can provide information for medical diagnostics. For example, researchers have proposed new techniques for determining blood flow (see documents [5], [6], [7]). Other authors have used the speckle phenomenon for the measurement of bone deformations or bone implants by interferometry (see documents [8] and [9]). Other authors have exploited the speckle for the determination of the roughness of surfaces and the surfaces of biological tissues (see document [10]). However, only the extraction of the roughness property restrains the analysis of the speckle, and the study of the tissular surface on its own is insufficient for a diagnostic assistance study. Indeed, the appearance of a pathology implies that physiopathological changes concern all the depths of a tissue and not only the surface of said tissue. In particular, the evolution of a pathological tissue generally begins by modifications at the level of the deepest layers and an early diagnostic then necessitates an exploration of the tissue of the deep layers of said tissue (dermis, hypodermis). Several researchers have also explored the relationship between the dimensions of the speckle and the experimental conditions (see document [11]).

However, a speckle coming from a living medium is dynamic and, from the point of view of signal processing, a conventional frequential approach seems not to be sufficient to study this non-stationary phenomenon. A fractal approach of the speckle has recently been introduced. In this approach, a parallel with fractional Brownian motion has been proposed (see document [12]). In the same way, other authors have proposed, by a dynamic study of the speckle coming from various samples (inert materials or biological samples such as fruits or plants), extracting the fractal dimension of speckle figures modelled as fractional Brownian motion, in order to characterise these different media (see document [13]).

Fractional Brownian motion is a stochastic process widely used in fractal approaches. Moreover, fractal approaches have recently been used for the characterisation of real complex phenomena. In the biomedical field, the works of Pothuaud (see document [14]) and Benhamou (see document [15]), who use fractals to analyse the bone textures of radiographic images, may be cited. Fractal properties have been found previously in the speckle phenomenon, namely in the speckle generated by a randomly rough surface (see document [16]) and in the speckle generated by calibrated polystyrene microsphere solutions (see document [12] and document [17]). The fractal approach of the speckle proposed by document [12] and a conventional frequential approach of the speckle have been used to characterise a pathological skin attacked by scleroderma at a stationary phase, the lesions of which were visible (see document [18]). However, the device used in document [18] has not proved to be efficient for the detection of radiological burns. This device does not make it possible, unlike the invention, to detect a pathology in evolution in which the lesions are not yet visible or when the physiopathological changes take place at depth.

In the present invention, the fractal approach of the speckle by the fractional Brownian motion, proposed in document [12], is applied to the discrimination, in vivo, of cutaneous pathologies.

According to document [12], there exists a parallel between the speckle phenomenon and the Brownian motion phenomenon. Indeed, their first order statistics are of the same nature: they are Gaussian for the amplitude distribution and for the intensity distribution. Their second order statistics also have the same characteristics: a power spectral density, designated PSD hereafter, showing a decrease in 1/f, where f is the frequency, and a Gaussian increment in both cases.

In the case of the speckle, the PSD of the experimental figures decreases according to a power law only in the high frequencies domain, which confirms an autosimilar behaviour (or scale invariance) in this spectral domain.

The generalisation to fBm, in other words to fractional Brownian motion, makes it possible to add an additional degree of freedom, which makes the model more flexible. This is why the generalisation to fractional Brownian motion has been considered. By virtue of this modelling, it is possible to extract three stochastic parameters characterising a speckle image from its diffusion function:
the Hurst coefficient H, characterising the fractal dimension of the image,
the size of the autosimilar element S, characterising the separation of autosimilar and conventional behaviours in the image, and
the saturation of the variance G, which gives the asymptotic direction at high neighbourhood values in the image.

A detailed description of the statistical theory of the speckle as well as the correlation between this phenomenon and that of fractional Brownian motion is given at the end of the present description.

A photographic plate simply has to be placed at any distance from the object to record the speckle. It may be observed either in “free space” (objective speckle) or on an image plane of the illuminated object (subjective speckle). In the first case, the speckle is recorded by a camera without objective and without any other imaging system and, in the second case, by a camera with an objective for example.

Any modification of the diffusing medium leads to optical and statistical modifications of said medium, which leads to the variation in the above mentioned three stochastic parameters.

The idea is then to use these parameters that characterise the speckle image with a view to differentiating the diffusing media. Since diagnostic assistance is an objective of the present invention, the application of this method is targeted on the living medium, in particular on the cutaneous syndrome of acute irradiation, the evolution of which in the short and the long term is still little known. This approach of the speckle phenomenon, based on the fractal theory, is more powerful than the conventional frequential approach (these two approaches are described at the end of the present description) since it integrates the multi-scale aspect of the speckle.

An observation and acquisition device of speckle figures according to the invention is described hereafter.

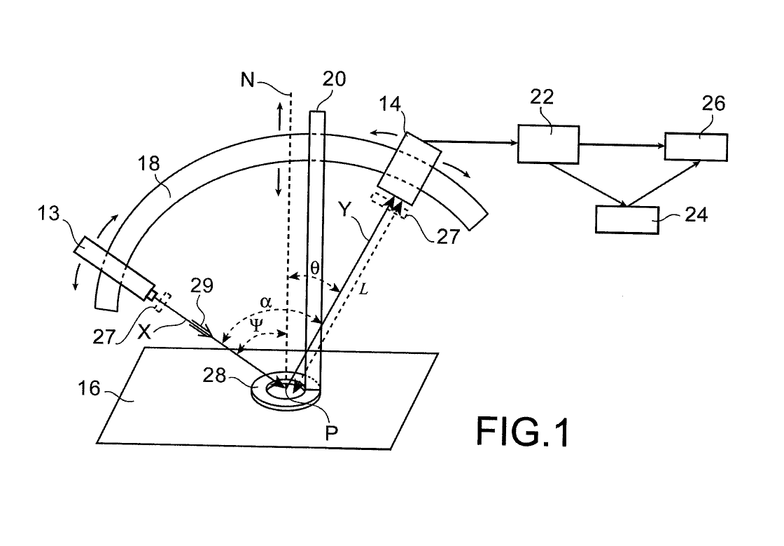

The device according to the invention, which is schematically represented in FIG. 1, is used to record the speckle fields coming from biological tissues. This device is very simple and not very expensive. It comprises a non-polarised monochromatic laser 13 and a charge coupled device camera 14, more simply known as “CCD camera”. A diffusing medium 16, namely a sound or pathological cutaneous area, illuminated at a point P by the beam 29 coming from the laser 13, generates a speckle phenomenon. The light back scattered by the medium (cutaneous tissue) 16 is captured by the camera 14, which thus enables the acquisition of a speckle.

N designates the direction of the normal to the surface of the biological tissue 16 at point P, X the direction of emission of the light by the laser 13, Y the direction of observation of the speckle field by the camera 14. The following angles without particular orientation are designated as follows: α the angle between the directions X and Y, Ψ the angle of incidence of the laser beam compared to the direction normal to the surface of the biological tissue (angle between the directions X and N) and θ the angle of observation compared to the direction normal to the surface of the biological tissue (angle between the directions Y and N). Δα designates the difference in absolute value between the two angles Ψ and θ and is known as the observation angle compared to the direction of the specular reflection. This thus gives Δα=|Ψ−θ|. This angle gives the difference in the direction of observation compared to that of the specular reflection: the more it increases, the more the observation differs from the specular reflection and thus the more the photons that have diffused into the deep layers of the medium are observed.

The device of FIG. 1, according to the invention, enables the variation of the angle Ψ (respectively θ) of the direction X (respectively Y) independently of the variation of the angle θ (respectively Ψ) of the direction Y (respectively X). For this purpose, the device of FIG. 1 also comprises mechanical means comprising a mechanical support 18 and a mechanical guide 20. The mechanical support 18 supports the laser 13 and the camera 14 and enables a variation of the angle Ψ and/or the angle θ, in order to observe the speckle field at different angles. This variation of angles Ψ and/or θ makes it possible to explore the tissue at various depths.

The lower part of the guide 20 is rigidly integral with a torus 28 that delimits the measurement area. Furthermore, said torus is in contact with the surface of the tissue 16. The internal diameter of the torus is equal to 40 mm in the example; it is then wide enough not to add parasitic reflections. The guide 20 and the torus 28 make it possible to maintain a constant distance L between the point of impact P of the laser beam 29 and the camera 14, between two consecutive speckle figure acquisitions, and also make it possible to absorb possible movements of the tissue 16, for example due to breathing. The guide 20 and the torus 28 then assure an optimal acquisition of the speckle figures for the indispensable comparison between the two areas (sound and pathological).

The mechanical support 18 is fixed to the guide 20 and adjustable in height on said guide 20, and this support forms an arc of circle, the direction of the curve radius of which attains substantially point P. The laser 13 and the camera 14 are fixed and adjustable in position on the support 18. It is thus possible to adjust the angle Ψ to a value of the interval ranging substantially from 0° to 90° and it is also possible to adjust the angle θ to a value of the interval ranging substantially from 0° to 90°.

Obviously, the length of the arc of circle shaped support 18 is chosen as a function of the maximum angle α that it is wished to obtain with the device: if it is desired to obtain an angle α substantially equal to 180°, a support 18 forming substantially a half-circle is used.

The device of FIG. 1 also comprises electronic means 22 to process, according to the invention, the signals provided by the camera. These electronic means 22 are provided with display means 26.

It is pointed out that, according to the invention, the tissue 16 is illuminated by means of the laser 13 in a sound area then in an area liable to comprise modifications.

The device of FIG. 1 further comprises electronic means 24 for analysing the signals which are processed according to the invention by the means 22, so as to validate the comparison of the two cutaneous areas (sound and pathological). The results obtained by these means 24 may also be displayed by the display means 26.

In the example, the laser 13 is a non-polarised He—Ne laser (632.8 nm) of 15 mW power, which emits a beam, the width of which is around 1 mm at I₀/e², where I₀ is the maximum intensity of the laser (radius of the beam for which the intensity has decreased by a factor 1/e² compared to its maximum I₀).

The CCD camera 14 is for example of the Kappa CF 8/1 DX type, with 376(H)×582(V) effective pixels; it is used without objective, and each pixel measures 8.6(H)×8.3(V) μm. The exposure time of the camera enables an exposure time at least equal to 100 μs.

In addition, it is pointed out that the camera is intended to acquire at least 200 speckle figures per area illuminated at a frequency of 25 Hz.

Furthermore it should be noted that, for the measurements, the laser 13 and the camera 14 are not necessarily placed on either side of the guide 20: if necessary, for these measurements, they can be on the same side of this guide.

A moving arm (not represented) maintains the mechanical support 18-guide 20 assembly, which supports the laser 13 and the camera 14, and enables their displacement to study different areas of the tissue 16. The displacement takes place in translation and/or in rotation in the three directions of space in order to adapt to the measurements of the different areas of the tissue 16 to be studied.

It is pointed out that the invention may be implemented with other observation and acquisition means than a CCD camera and that said CCD camera and the other cameras used may be provided, or not, with an objective for the implementation of the invention. In the same way, the invention can also be implemented with a polarised laser.

Furthermore, the selection of the speckle coming from deep or surface layers of the tissue may be completed by an optical system 27, constituted of polarisers (linear, circular, or elliptical) and/or half or quarter wave plates. This optical system, when it is used, is placed at the output of the laser and/or at the input of the camera. This optical system makes it possible to control the polarisation of the coherent light illuminating the tissue and the polarisation of the light arriving on the camera in order to detect several polarisation states according to the polarisation configuration chosen at the output of the laser. The polarisers with or without half or quarter wave plates are configured in order to select preferentially the speckle coming from surface layers of the tissue or the speckle coming from more or less deep layers.

The cutaneous effects of the acute irradiation cutaneous syndrome in several pigs were taken as examples of application of the device according to the invention: the pigs were irradiated locally (40 Gy) by gamma radiation on their right sides, over an area of dimension 5 cm×10 cm.

In an example of the invention, the speckle figures obtained by successively illuminating the two areas (sound and pathological), at several angles Ψ ranging from 20° to 60° and by detecting the light back scattered at a fixed angle θ, chosen equal to 0°, are processed; this processing is carried out by a conventional frequential method and a fractal method: the CCD camera 14 provides electrical signals representative of the speckle figures and the electronic processing means 22 process these signals by the two methods cited above, in the form of non-compressed images, and make it possible to compare the two areas. This comparison is validated by the electronic statistical analysis means 24 (statistical tests such as Student tests and the analysis tests of the variance, or factorial analyses such as, for example, Principal Component analysis).

The recording of the speckle figures necessitates a few precautions.

Indeed, the speckle studied is produced by a living medium that consequently contains mobile diffusers, the movement of which may be considered as random. This leads to an agitation of the speckle, known as “boiling speckle”, which corresponds to temporal fluctuations in the intensity of the speckle. These temporal fluctuations are normally described by the temporal autocorrelation function of the intensity (see document [19]).

As a result, the acquisition time of a speckle image must be as short as possible in order to avoid recording this “scrambled” speckle. Since the camera enables a variable exposure time, the shortest acquisition time, equal to 100 μs, is chosen despite any loss of a correct signal to noise ratio.

In addition, the size of the speckle grains increases linearly with distance (see document [20]). Also, the speckle grains recorded have to be quite large compared to the size of the pixels of the CCD camera, which implies that said camera must not be too near to the diffusing medium. In addition, each image has to contain enough grains to carry out a significant statistical study of each image, which means the camera must not be too far either from the medium.

It is difficult to find the distance L between the CCD sensor and the illumination point of the diffusing medium by respecting these conditions ideally. A compromise must thus be found. The distance L chosen was 20 cm for pig skin. This choice is provided purely by way of indication and is in no way limiting.

Nevertheless, the distance L must be identical for the first and second areas, in other words the sound area and the area likely to comprise lesions.

In order to avoid the direct recording of the light of the laser that is directly reflected by the surface of the medium (specular reflection), and thus in order to avoid the saturation of the camera sensor, the observation and the acquisition of the speckle field take place outside of the specular reflection at more or less 10°.

A series of images is recorded by the CCD camera with a frequency of 25 Hz. A complete video image is composed of two fields acquired one after the other: an even field (composed of even lines 2, 4, 6. etc.) and an odd field (composed of odd lines 1, 3, 5, etc.). Thus, 50 fields (even and odd) will be delivered per second to obtain a complete image at a frequency of 25 Hz. Once again, given the dynamic nature of the speckle, the images are acquired on a unique field (even or odd) since the image changes between the acquisition of an even field and an odd field. The dimensions of an image are thus 288×384 instead of 576×384 for a complete non-compressed image.

The analogue signal delivered by the camera is then digitised on 8 bits by an image acquisition card, which makes it possible to measure the intensity on a grey level scale going up to 256.

In order not to have any loss or any deformation of the information contained in the digital signal, no compression takes place.

The number of images acquired is 200 per measurement point (corresponding to the impact point of the laser beam P) at a frequency of 25 images per second and with an acquisition time of 100 μs. Several measurements points are taken for each area analysed of skin (sound area and pathological area).

The speckle images are then processed to determine the “speckle size” (average size of the grains of a speckle image), by a conventional frequential method, recalled at the end of the present description.

The images are also processed line by line or column by column, by a fractal method, so as to determine the three stochastic coefficients thereof as indicated at the end of the description. For one image and for each dimension of the image (horizontal or vertical), a calculated stochastic coefficient (Hurst coefficient. H, saturation of the variance G or autosimilarity S) along the horizontal dimension (respectively vertical) corresponds to the average of the coefficients found for each diffusion curve corresponding to each line (respectively column) of the image. It is thus possible to compare the results obtained by the two methods.

The application of the invention to cutaneous irradiation in pigs will now be considered.

According to the device of FIG. 1, according to the invention, for a constant angle θ, the greater the angle of incidence of the laser beam Ψ, the greater the diffusing surface and volume. In the same way, for a constant angle Ψ, the diffusion surface and volume are observed differently according to the position of the camera in the plane of the observation: the greater the angle θ between the direction of the observation and that of the normal to the surface of the tissue, the greater the diffusing surface and volume observed by the camera. Moreover, the greater the angle Ψ with respect to θ or inversely the greater the angle θ with respect to Ψ, the less the energy flow captured by the camera takes into account the specular reflection. Thus, the probability of taking into account multidiffused photons, those coming from deeper layers of the skin, increases with the difference in absolute value between the two angles Ψ and θ. It is recalled that this difference of angles, in absolute value, is designated Δα and that it corresponds to the angle of observation compared to the direction of the specular reflection. Consequently, the more one moves away from specular reflection, the greater the probability that these measurements contain information coming from the volume; the information coming from the deep layers then predominate over that coming from the surface. However, unlike the case of a pathology at an advanced stage, as was the scleroderma (see document [18]), in the case of a pathology in evolution for which the lesions are not necessarily visible (radiological burn during the clinically silent phase for example), the physiopathological changes of the tissue take place first at depth; an efficient diagnostic is then based on the observation of these changes at these depth scales. Thus, with the intention of taking into account, in the recording of the speckle field, the cutaneous changes taking place at different layers and different cutaneous depths, it is necessary, for an efficient and reliable diagnostic assistance, to observe the speckle field under different angles compared to the direction of the specular reflection (Δα variable and greater than 10°). To do this, the arc shaped mechanical component 18 (FIG. 1) supporting the laser 13 and the camera 14, has been considered in the constitution of the device of FIG. 1, according to the invention, in order to enable a variation of the angles Ψ and/or θ and thus of the angle Δα=|Ψ−θ|, and thus to make it possible to record the speckle generated by the skin at various depths: an angle Δα of 20° being linked to the information contained essentially in the superficial layers and an angle Δα of 60° to the information contained essentially in the deep layers such as the deep dermis or the hypodermis.

In the radiological burn application, it has been chosen to carry out the measurements with a value of the angle of incidence of the laser beam Ψ in the interval ranging from 20° to 60° and a fixed value of the angle θ, chosen equal to 0°. In this application, the angle of observation of the speckle compared to the direction of the specular reflection Δα was then of a value equal to that of the angle Ψ.

A pre-clinical study model was developed specifically for the application of the invention to cutaneous irradiation in pigs. It involves a calibrated localised irradiation model in pigs, simulating in a reproducible manner radiological burns in humans.

Pig skin is the best known biological model for human skin. The irradiations take place by gamma radiation (⁶⁰Co, 1 Gy/minute). During the irradiation, the pig is laid on its belly and arranged so that the axis of the irradiation beam is perpendicular to the axis of its spinal column. A block of wax of around 1 cm thickness is placed on the area of irradiated skin in order to assure electronic equilibrium conditions at the level of the skin and thus to obtain a better uniformity of the dose in depth. Thermoluminescent dosimeters, constituted of alumina powder (Al₂O₃), are incorporated in the thickness of wax in order to control the dose delivered on the skin.

This experimental irradiation protocol has been validated by a series of measurements on a simplified phantom, representative of the principal characteristics of the pig (thickness and height of the trunk, density of the skin).

Irradiations were carried out by following this experimental protocol, at different doses, namely 5, 10, 15, 20, 40 and 60 Gy and made it possible, under these experimental conditions, to select the dose of 40 Gy, the dose at which signs of necrosis have been observed. By observing the evolution of the clinical signs of the radiological burn in a first pig irradiated at 40 Gy, it is possible to see an evolution similar to that which is observed in humans with a latency phase that precedes necrosis. In the case of the above mentioned pig, this latency phase goes from D3 to D104, in other words from 3 days to 104 days after the day of irradiation, which is designated D0. In clinical terms, a slight temporary erythema was observed twenty four hours after the irradiation; it is confirmed at D2 and disappears as of D3.

By observing the evolution of the irradiated area by the Doppler laser technique, a difference in cutaneous response is observed with a hypervascularisation image corresponding to the development of the inflammatory reaction (erythema), principally at D1. This reaction subsides at D2 to disappear as of D3. No image made it possible to distinguish the irradiated area up to the end of the experiment. In fact, it is observed that the images of the Doppler technique are significant only when the erythema is visible, at D1 and at D2.

According to these defined experimental conditions, it was decided to apply the exploitation of the statistic of the speckle field to this animal model by the device object of the invention.

The experimental protocol that has been chosen for the exploitation of the statistic of the speckle field coming from pig skin is given hereafter.

Four irradiations by gamma radiation (⁶⁰Co) were carried out locally on the pig skin, on a surface of 5 cm×10 cm with a dose of 40 Gy.

Series of measurements were carried out around every 8 days after irradiation. Eight measurement points were taken on each area (sound area, corresponding to 0 Gy, and irradiated area corresponding to 40 Gy) with 200 images for each point. In order to be certain of measuring at each experiment at the same place on this skin, the skin was tattooed on each area (sound and irradiated) so as to delimit 8 squares of 1 cm². The measurements were thus carried out for around 3 to 4 months. At each experimentation date and for each measurement point, there is a large sample size (n=200). With the aim of comparing the variability between the measurement points for a same area and the variability between areas, the two factor ANOVA test (see document [21]) was applied. The parameter p_{A}, the p-value for the null hypothesis H_{0A}, corresponding to the factor A (inter-area variability), and the parameter p_{B}, the p-value for the null hypothesis H_{0B}, corresponding to the factor B (variability intra-area) are defined. Comparisons between the sound and irradiated areas were then validated at each experimentation date by the above mentioned statistical test. At the end of the measurement campaign, the areas measured were biopsied for a histological validation of the measurements.

The device of FIG. 1 was used in the case of radiological burns and the experimental context was as follows:
constant distance between the CCD camera and the illumination point P of the skin: L=20 cm;
angle of incidence of the laser beam in relation to the direction normal to the surface taking the following values: ψ=20°, 40° and 60°;
observation angle of the camera in relation to the direction normal to the surface chosen as fixed: θ=0°;

under these experimental conditions, the angle of observation of the speckle compared to the direction of the specular reflection Δα is then equal to the angle ψ; hereafter, in this application, we will then mix up the designation of these two angles;
acquisition time of an image:100 μs; and
the images have not been compressed.

The example of a pig numbered P129 will now be considered.

1. Conventional Frequential Approach: Calculation of the Size of Grains.

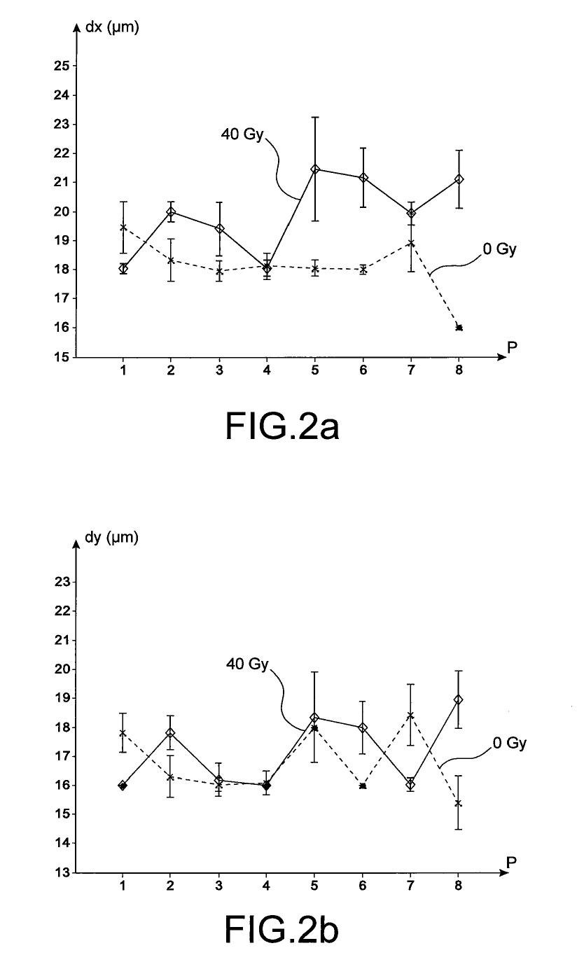

The images were all processed but, for reasons of clarity, only the numerical and graphical results at D64 after irradiation and for ψ=20° are represented here, shown in table 2 and FIGS. 2a and 2b.

By using the ANOVA test described previously, one obtains for the width dx of grains (FIG. 2a): p_{A}=0.044 and p_{B}=0.93; for the height, or length, dy of the grains (FIG. 2b): p_{A}=0.57 and p_{B}=0.82. By taking a threshold of 0.01 for the value of p, no discrimination between 0 Gy and 40 Gy is possible by the calculation of the size of the grains.

In the same way, the results corresponding to other measurements (other dates and other angles of incidence of the laser beam) show a similar behaviour with values of p_{A} ranging between 0.13 and 0.93 for more than 8 cases out of 10 and between 0.029 and 0.13 for less than 2 cases out of 10.

TABLE 2
Results for the average size of
grains, for each measurement point P and for each area:
sound (0 Gy, dotted lines in FIGS. 2a and 2b) and
irradiated (40 Gy, solid lines in FIG. 2a and 2b) for
the pig P129 at D64 and for ψ = 20°

| | Point 1 | Point 2 | Point 3 | Point 4 | Point 5 | Point 6 | Point 7 | Point 8 |
|---|---|---|---|---|---|---|---|---|
| 0 Gy | | | | | | | | |
| Width | 19.45 ± 0.9 | 18.3 ± 0.73 | 17.93 ± 0.35 | 18.11 ± 0.45 | 18.04 ± 0.28 | 17.98 ± 0.17 | 18.89 ± 0.99 | 16 ± 0.03 |
| dx (μm) | | | | | | | | |
| Height | 17.81 ± 0.68 | 16.3 ± 0.71 | 16.02 ± 0.22 | 16.09 ± 0.41 | 18 ± 0.02 | 16 ± 0.03 | 18.42 ± 1.05 | 15.4 ± 0.92 |
| dy (μm) | | | | | | | | |
| 40 Gy | | | | | | | | |
| Width | 18.01 ± 0.17 | 19.99 ± 0.34 | 19.39 ± 0.92 | 18.04 ± 0.28 | 21.45 ± 1.03 | 21.15 ± 1.03 | 19.92 ± 0.4 | 21.09 ± 0.99 |
| dx (μm) | | | | | | | | |
| Height | 16 ± 0.03 | 17.81 ± 0.58 | 16.18 ± 0.57 | 16 ± 0.03 | 18.34 ± 1.56 | 17.98 ± 0.9 | 16.02 ± 0.22 | 18.95 ± 1 |
| dy (μm) | | | | | | | | |

2. Fractal Approach: Calculation of the Three Stochastic Parameters.

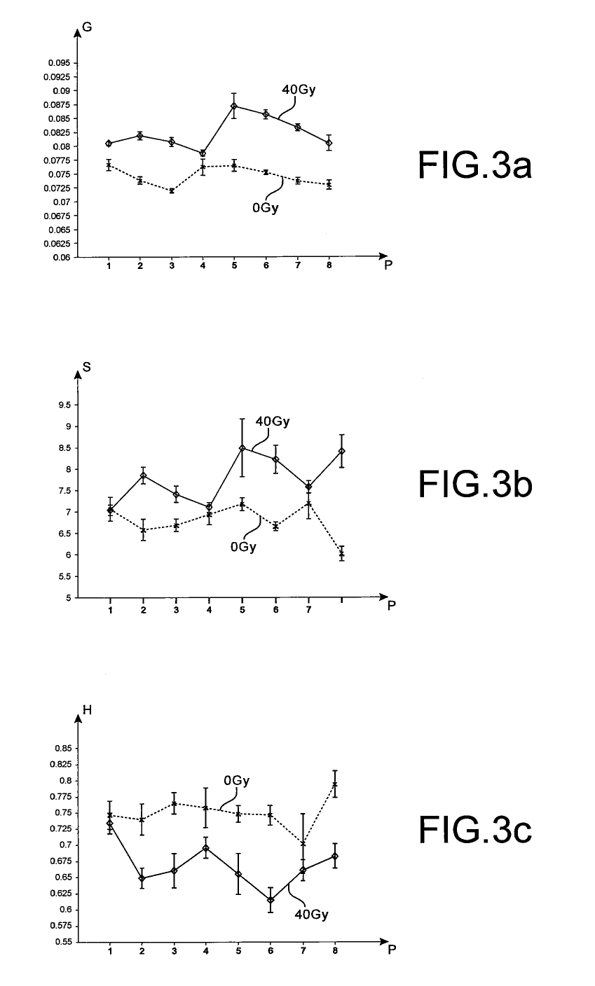

In the same way, for reasons of clarity, only the numerical and graphical results at D64 after irradiation are represented, for an angle of incidence of the laser beam of ψ=20° and for the horizontal dimension of the image, although the images were all processed. These results are shown in table 3 and FIGS. 3a, 3b and 3c.

TABLE 3
Results of the stochastic approach
for the speckle for each measurement point P and for
each area: sound (0 Gy, dotted lines in FIG. 3a, 3b
and 3c) and irradiated (40 Gy, solid lines in FIGS.
3a, 3b and 3c) for the pig P129 at D64, for ψ = 20° and
for the horizontal dimension of the image

| | Point 1 | Point 2 | Point 3 | Point 4 |
|---|---|---|---|---|
| 0 Gy | | | | |
| Saturation | 0.0766 ± 0.0011 | 0.074 ± 0.0006 | 0.0719 ± 0.0004 | 0.0762 ± 0.0015 |
| of the | | | | |
| variance | | | | |
| (G) | | | | |
| Autosimilarity | 7.06 ± 0.27 | 6.56 ± 0.25 | 6.67 ± 0.14 | 6.92 ± 0.23 |
| (S) | | | | |
| Hurst (H) | 0.746 ± 0.022 | 0.74 ± 0.024 | 0.765 ± 0.0169 | 0.758 ± 0.03 |
| 40 Gy | | | | |
| Saturation | 0.0805 ± 0.0004 | 0.0818 ± 0.0007 | 0.0807 ± 0.0008 | 0.0787 ± 0.0005 |
| of the | | | | |
| variance | | | | |
| (G) | | | | |
| Autosimilarity | 7.03 ± 0.12 | 7.85 ± 0.2 | 7.4 ± 0.19 | 7.09 ± 0.11 |
| (S) | | | | |
| Hurst (H) | 0.734 ± 0.016 | 0.649 ± 0.016 | 0.661 ± 0.026 | 0.696 ± 0.016 |
| | Point 5 | Point 6 | Point 7 | Point 8 |
| 0 Gy | | | | |
| Saturation | 0.0765 ± 0.0011 | 0.0752 ± 0.0004 | 0.0736 ± 0.0006 | 0.073 ± 0.0008 |
| of the | | | | |
| variance | | | | |
| (G) | | | | |
| Autosimilarity | 7.17 ± 0.15 | 6.65 ± 0.1 | 7.19 ± 0.36 | 6.01 ± 0.17 |
| (S) | | | | |
| Hurst (H) | 0.748 ± 0.013 | 0.746 ± 0.015 | 0.702 ± 0.046 | 0.79 ± 0.021 |
| 40 Gy | | | | |
| Saturation | 0.0872 ± 0.0022 | 0.0856 ± 0.0008 | 0.0833 ± 0.0006 | 0.0805 ± 0.0014 |
| of the | | | | |
| variance | | | | |
| (G) | | | | |
| Autosimilarity | 8.48 ± 0.68 | 8.21 ± 0.33 | 7.57 ± 0.14 | 8.4 ± 0.38 |
| (S) | | | | |
| Hurst (H) | 0.655 ± 0.031 | 0.615 ± 0.019 | 0.661 ± 0.016 | 0.683 ± 0.019 |

The two factor ANOVA test gives the following values for the index p:
saturation of the variance G (FIG. 3a): p_{A}=0.002 and p_{B}=0.29
autosimilarity S (FIG. 3b): p_{A}=0.011 and p_{B}=0.84, and
Hurst H (FIG. 3c): p_{A}=0.0007 and p_{B}=0.31.

The discrimination between the sound area and the irradiated area is then significant to more than 99.8% for the Hurst coefficient and for the saturation of the variance. The autosimilarity is “nearly” discriminating if a threshold of 0.01 is taken for the index p_{A}. However, it is the only series of measurements where its index is as low, since for all the other measurements (corresponding to the other angles of incidence of the laser beam and at other dates) the index p_{A} was too high for the discrimination (p_{A}>0.023).

On the other hand, the Hurst coefficient always makes it possible to discriminate the irradiated area from the sound area from D64 for ψ=20° (see table 4).

The parameters calculated along the horizontal dimension of the image have discriminated in the same way, for each experimentation date and each angle, as those calculated along the vertical dimension of the image.

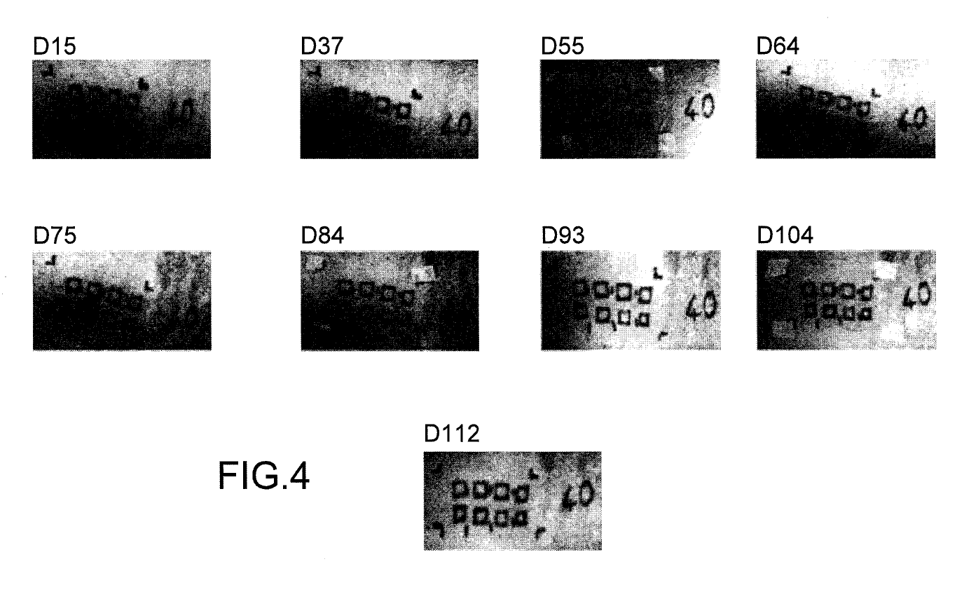

FIG. 4 shows photographs of the pig skin (irradiated area) at all the measurement dates.

TABLE 4
Parameters calculated for the
horizontal dimension of the image (saturation of the
variance G, autosimilarity S and Hurst H and width of
the grain dx) discriminating for the three angles of
observation studied (20°, 40°, 60°) and clinical
expression of the irradiated cutaneous tissue for all
of the measurement dates

| Discriminating | | | | |
|---|---|---|---|---|
| coefficients | | | | Visible clinical sign |
| (p ≦ 0.01) | Ψ = 20° | Ψ = 40° | Ψ = 60° | (vcs) |
| D15 | — | — | — | None (vcs) |
| D37 | — | — | — | None (vcs) |
| D55 | — | — | — | None (vcs) |
| D64 | H, G | H, G | H, G | None (vcs) |
| D75 | H, G | H, G | H, G | None (vcs) |
| D84 | H | G | H | None (vcs) |
| D93 | H | H | — | None, suffering |
| | | | | (vcs) |
| D104 | H | — | H | None (vcs) |
| D112 | H | — | H | None, suffering (vcs) |

As may be seen in table 4 and in the photographs of FIG. 4, despite the absence of any visible lesion (erythema or other), the Hurst coefficient H and the saturation of the variance G discriminate the irradiated area at D64 and D75 for the three observation angles. From D84, only the Hurst coefficient discriminates at least for two of the three angles. This coefficient is more efficient for the discrimination.

It should be noted that the animal has a considerable sensitivity of the irradiated area to the touch at D93, which constituted the first clinical sign. The appearance of surface pain is generally considered as predictive of the appearance of a necrosis in humans. It may be noted that the discrimination for the 3 angles chosen appears before this pain phase (D64, D75, and D84).

The example of three other pigs numbered P161, P163 and P164 is now considered.

The results for these three other pigs (P161, P163 and P164) are shown in table 5 in the form of scores of discriminating parameters (G, H, S and dx), scores made on all of the measurement dates and for each angle measured. The parameters are represented in table 5 for the horizontal dimension of the image. As for the pig P129, for each experimentation date and for each angle, the discrimination was not different with the parameters calculated along the vertical dimension of the image.

Discrimination was possible during the clinically silent phase when no clinical sign was yet visible, and the first discrimination during this phase was made thus:
Pig P161: 20 days before the appearance of the first lesion and by the parameters H, G at ψ=60°
Pig P163: 57 days before the appearance of the first lesion and by H at ψ=60°
Pig P164: 56 days before the appearance of the first lesion and by H at ψ=20° and 60°.

It may be noted that the angle ψ=60° enables the first discrimination for these three pigs, the first modifications of the tissue, due to the irradiation, then seem to take place within the deep layers.

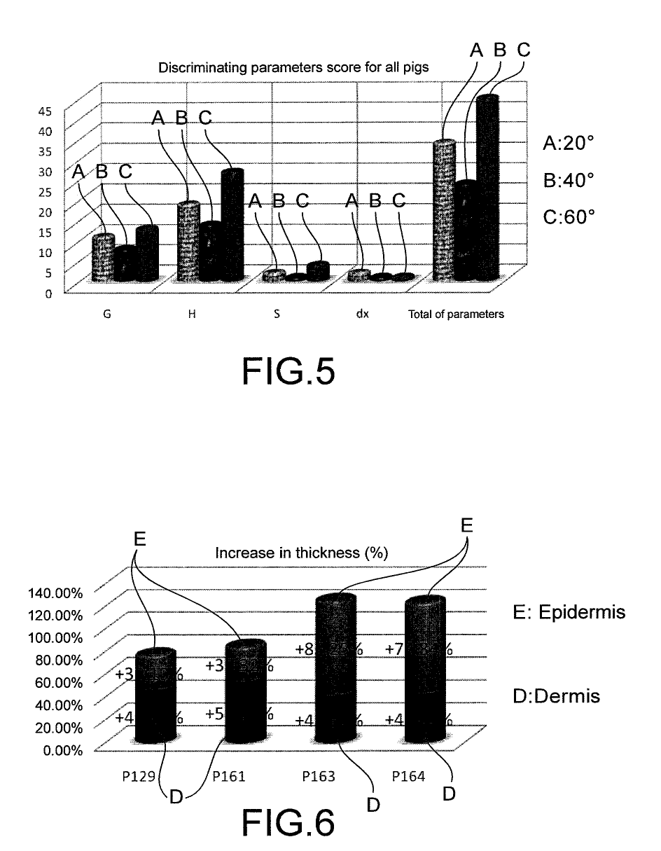

FIG. 5 is a graphical representation of the scores of discriminating parameters for each angle, all pigs taken together (pigs P129, P161, P163 and P164).

It may also be observed that, for all of the pigs, the Hurst parameter is the most efficient for the discrimination and that ψ=40° is the least efficient angle (FIG. 5 and table 5) particularly for an early discrimination. The high efficiency of the observation angle ψ=60° implies that the physiopathological changes take place essentially in the deepest layers of the skin. The efficacy of the diagnostic, in the case of radiological burns, is then based on the observation of the deepest cutaneous layers. The efficacy of the angle ψ=20° indicates that important modifications also take place in the superficial layers of the skin (epidermis). The intermediate layers, visible essentially at 40°, would not be subject to important modifications in the case of radiological burns, which would explain the poor efficacy of this angle for the discrimination. Consequently, in order to take into account the physiopathological changes situated at various cutaneous depths and thus not to neglect any cutaneous layer where modifications leading to variations of the speckle field could take place, it is necessary to explore the whole cutaneous depth for an optimum diagnostic and as early as possible; this is possible by varying the angle of observation Δα compared to the specular reflection.

TABLE 5
Scores on all of the
experimentation dates of discriminating parameters
calculated along the horizontal dimension of the image
(three stochastic parameters (saturation of the
variance G, autosimilarity S and Hurst H) and width of
the grains dx) for each observation angle and for each
pig. The total of the scores for all of the pigs is
also indicated.

| | Discriminating parameter | | | | | | | | | | | |
|---|---|---|---|---|---|---|---|---|---|---|---|---|
| | Ψ = 20° | | | | Ψ = 40° | | | | Ψ = 60° | | | |
| Pig | G | H | S | dx | G | H | S | dx | G | H | S | dx |
| P129 | 2 | 6 | 0 | 0 | 3 | 3 | 0 | 0 | 2 | 5 | 0 | 0 |
| | Total = 8 | | | | Total = 6 | | | | Total = 7 | | | |
| P161 | 1 | 4 | 2 | 2 | 2 | 2 | 1 | 1 | 3 | 7 | 2 | 0 |
| | Total = 9 | | | | Total = 6 | | | | Total = 12 | | | |
| P163 | 5 | 6 | 0 | 0 | 2 | 4 | 0 | 0 | 3 | 7 | 0 | 0 |
| | Total = 11 | | | | Total = 6 | | | | Total = 10 | | | |
| P164 | 3 | 3 | 0 | 0 | 1 | 5 | 0 | 0 | 5 | 8 | 2 | 1 |
| | Total = 6 | | | | Total = 6 | | | | Total = 16 | | | |
| Total for all of | 11 | 19 | 2 | 2 | 8 | 14 | 1 | 1 | 13 | 27 | 4 | 1 |

| | | | |
|---|---|---|---|
| the pigs | Total = 34 | Total = 24 | Total = 45 |

FIG. 6 shows the increase in the thickness of the epidermis and the thickness of the dermis of the irradiated area compared to the sound area (in %) for the four pigs.

The histology on the biopsy of sound and irradiated areas makes it possible to quantify the level of damage of the cutaneous tissue and to correlate the evolution of the physical parameters with the corresponding biological modifications. The histological measurements carried out at D112 for the pig P129, at D106 for the pig P161, at D92 for the pig P163 and at D168 for the pig P164 show an increase in the thicknesses of the epidermis and the dermis of:

30% and 47% respectively for the pig P129

30% and 54% respectively for the pig P161

83% and 42% respectively for the pig P163

80% and 43% respectively for the pig P164.

Table 6 shows the correlation coefficients (r) calculated between the parameters of the speckle, calculated along the horizontal dimension of the image (G, S, dx and H), and the thicknesses of the epidermis and the dermis. The correlation calculations were carried out by considering all of the measurement points and all of the four pigs studied. The significance of the test carried out on the correlation coefficient is also indicated, with a threshold of the confidence index p chosen here to be 0.005. The symbol ˜ signifies “little different from”.

The calculations of the correlations between the different thicknesses and the parameters of the speckle (G, H, S and dx) show that the speckle is linked to changes of the dermis at ψ=40° and more strongly at ψ=60° by the Hurst parameter (Table 6). The exploration in depth of the skin by the device, object of the invention, is then confirmed by the Hurst parameter. Thus, the variation of the observation angle compared to the direction of the specular reflection, makes it possible, in the recording of the speckle field, to take into account different cutaneous layers, from the surface layers to the deepest layers, and thus enables an early diagnostic and the localisation of the physiopathological cutaneous changes leading to modifications of the speckle observed.

TABLE 6
Correlation coefficients (r)
calculated between the parameters of the speckle (G, S,
dx and H), calculated along the horizontal dimension of
the image, and the thicknesses of the epidermis and the
dermis. The significance of the test carried out on the
correlation coefficient is also indicated, with a
threshold for the confidence index p chosen here of
0.005

| Ψ = 20° | | Ψ = 40° | | Ψ = 60° | |
|---|---|---|---|---|---|
| G/ | | G/ | | G/ | |
| Epidermis | G/Dermis | Epidermis | G/Dermis | Epidermis | G/Dermis |
| r = 0.443 | r = 0.240 | r = 0.511 | r = 0.141 | r = 0.413 | r = 0.061 |
| Significant | Not | Significant | Not | Significant | Not |
| | significant | | significant | | significant |
| | (p~0.1) | | (p~0.25) | | (p~0.65) |
| S/ | | S/ | | S/ | |
| Epidermis | S/Dermis | Epidermis | S/Dermis | Epidermis | S/Dermis |
| r = 0.324 | r = −0.101 | r = 0.351 | r = 0.181 | r = 0.382 | r = 0.262 |
| Significant | Not | Significant | Not | Significant | Not |
| | significant | | significant | | significant |
| | (p~0.45) | | (p~0.15) | | (p~0.05) |
| dx/ | | dx/ | | dx/ | |
| Epidermis | dx/Dermis | Epidermis | dx/Dermis | Epidermis | dx/Dermis |
| r = 0.387 | r = −0.210 | r = 0.377 | r = −0.051 | r = 0.436 | r = 0.142 |
| Significant | Not | Significant | Not | Significant | Not |
| | significant | | significant | | significant |
| | (p~0.1) | | (p~0.70) | | (p~0.30) |
| H/ | | H/ | | H/ | |
| Epidermis | H/Dermis | Epidermis | H/Dermis | Epidermis | H/Dermis |
| r = −0.736 | r = −0.282 | r = −0.683 | r = −0.402 | r = −0.674 | r = −0.592 |
| Significant | Not | Significant | Significant | Significant | Significant |
| | significant | | | | |
| | (p~0.025) | | | | |

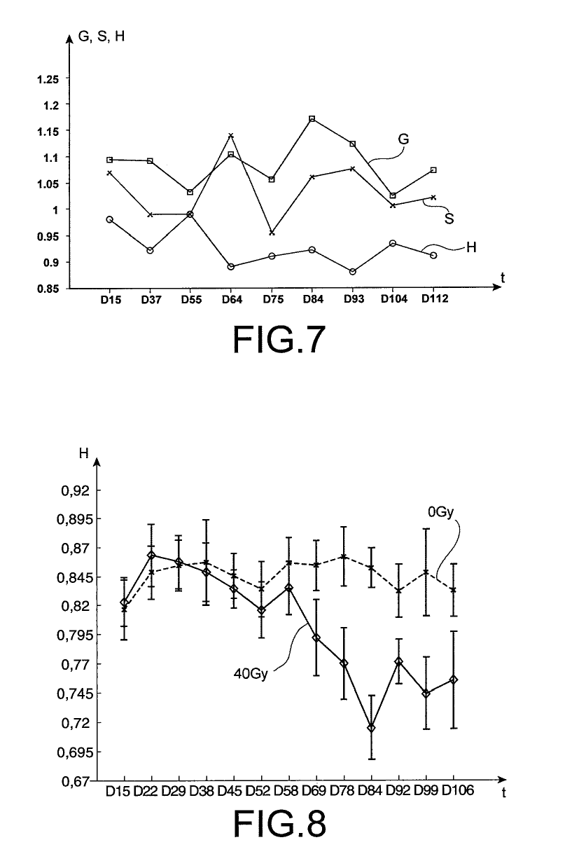

Table 7 and FIG. 7 show the evolution over time t of the ratio 40 Gy/0 Gy for the pig P129, for the angle of observation of ψ=20°, all measurement points taken together, for the three stochastic coefficients calculated along the horizontal dimension of the image.

It may be noted that, for all of the dates, the Hurst coefficient is lower for the irradiated area, unlike the saturation of the variance which, for its part, is higher. In addition, an overall reduction of this ratio for the Hurst coefficient may be observed as a function of time, which shows that it is the most efficient stochastic coefficient for the discrimination, as has been stated above.

TABLE 7
40 Gy/0 Gy ratio for the three
stochastic coefficients calculated along the horizontal
dimension of the image: saturation of the variance G,
autosimilarity S and Hurst coefficient H, for all of
the measurement dates and for ψ = 20°, all measurement
points taken together.

| | Ratio 40 Gy/0 GY | | | | | | | | |
|---|---|---|---|---|---|---|---|---|---|
| | D15 | D37 | D55 | D64 | D75 | D84 | D93 | D104 | D112 |
| Saturation of | 1.094 | 1.0921 | 1.032 | 1.103 | 1.055 | 1.17 | 1.122 | 1.024 | 1.071 |
| the variance | | | | | | | | | |
| (G) | | | | | | | | | |
| Autosimilarity | 1.069 | 0.99 | 0.99 | 1.14 | 0.955 | 1.06 | 1.075 | 1.004 | 1.02 |
| (S) | | | | | | | | | |
| Hurst (H) | 0.98 | 0.922 | 0.99 | 0.89 | 0.91 | 0.922 | 0.88 | 0.934 | 0.91 |

The evolution of the Hurst coefficient, calculated along the horizontal dimension of the image, is represented in FIG. 8, as a function of different dates of taking measurements after irradiation, for the sound area and the irradiated area, all measurement points taken together, for the pig P161 and ψ=60°, (dotted lines: 0 Gy, sound area; solid line: 40 Gy, irradiated area).

A fractal approach of the speckle phenomenon has been used for the discrimination of an inert medium, composed of latex beads of different concentration (see document [12]).

In the present invention, this stochastic approach is used for the purpose of making a diagnostic assistance device for radio-induced cutaneous lesions. The speckle field acquisition device, which is simple and not very expensive (FIG. 1), the measurement protocol, the processing of these speckle figures by a fractal approach and by a conventional frequential approach described at the end of the present description and the analysis of the processing of these figures by statistical methods making it possible to validate the comparison made between the sound and pathological areas according to the invention, are advantageous devices for, in vivo, diagnostic assistance for this pathology and the prognostic of its evolution.

In addition, the fractal approach used has proved to be more efficient for an early discrimination of the two areas (sound and irradiated); a fractal approach then appears more powerful for characterising speckle figures in a significant manner.

Moreover, it has been shown that the device represented in FIG. 1 has made it possible to discriminate the sound area from the irradiated area during the clinically silent phase by at least one of the three angles of observation used: 29 days before the appearance of the first lesion for the pig P129, 20 days for the pig P161, 57 days for the pig P163 and 56 days for the pig P164. The fact of being able to make a discrimination of the irradiated area, even though no lesion is visible, constitutes a very important and innovative factor. In addition, the non-invasive observation of the biological tissue at various depths, highlighted by the preceding correlations studies, makes it possible to reveal the localisation of physiopathological changes corresponding to the observed modifications of the speckle. In particular, significant variations alone of the Hurst parameter at ψ=60° correspond to changes at the level of the dermis and significant variations of one of the parameters or all of these parameters at ψ=20° correspond to changes at the level of the epidermis. The non-invasive exploration of the biological tissue at various depths and enabling diagnosis and prognosis, even though no clinical sign is visible, constitutes a very important and innovative factor.

In the examples given above, the invention has been implemented by carrying out the processing of speckle figures both by a conventional frequential method and by a fractal method. However, it would not go beyond the scope of the invention to carry out said processing simply by a conventional frequential method or by a fractal method or even by any other appropriate method.

Furthermore, returning to the device of FIG. 1, it is pointed out that the torus 28, placed at the base of the guide 20, may be replaced by any other means of delimiting the studied surface, since these means enable the laser beam 29 to attain this surface and also enables the back scattered light to be detected. Furthermore, the mechanical means constituted by the support 18 and the guide 20 may be replaced by other non-mechanical means having the same functions, for example mechanical-optical, acoustical-optical or electro-optical means.

In addition, it is pointed out that all of the components of the device of FIG. 1 are commercially available.

The invention enables not only pre-lesion discrimination, but also makes it possible to obtain a prognostic system for radio induced lesions and a mapping of the dose of the analysed tissue.

Moreover, the invention may be used in the context of a wider biomedical applications field than that of the diagnostic and the prognostic of the acute irradiation cutaneous syndrome. The numerous possibilities of biomedical applications may then be cited:
use as diagnostic assistance device for cutaneous lesions in general (cancer, local scleroderma, vitiligo, mycoses, etc.),
use as diagnostic assistance device for radio-induced lesions brought about by radiotherapy,
use as diagnostic assistance device for lesions caused by accidental irradiation,
use as diagnostic assistance device for lesions caused by burns other than those due to irradiation (thermal, chemical, electrical burns, solar erythema, etc.)
use for the prognostic of cutaneous lesions in general (radiological, thermal, chemical, electrical burns, etc., local scleroderma, skin cancer, etc.)
finally, in a much more general manner, use for the diagnosis and the prognosis of tissular lesions (cutaneous lesions, mucous lesions, systemic scleroderma, cancer, etc.).

Furthermore, the invention has two areas of application in the cosmetological field:
use for the evaluation of cutaneous ageing, and
use for the evaluation of the cosmetological or pharmacological efficacy of formulations or preparations for dermatological purposes.

The interest of the invention, is, on the one hand, that it makes it possible to detect an effect before said effect is visible and, on the other hand, that it represents a diagnostic assistance device that can be used in vivo and is above all non-invasive. The low cost of the device, object of the invention, facilitates its miniaturisation with the aim of making it an easily transportable tool for transfer in clinics and for distribution in hospitals.

The statistical theory of the speckle is recalled hereafter.

Goodman (see document [22]) and Goldfisher (see document [23]) were the first to study the statistical properties of the speckle and to express the power spectral density (PSD) and its autocorrelation function. The first and the second order statistics of the speckle are described hereafter.

First Order Statistic

Let us consider a coherent light beam back scattered by a diffusing surface. At each point of space, the amplitude of the electrical field corresponds to the sum of contributions in amplitude of the different surface diffusers:

$$A ( x , y , z ) = \frac{1}{\sqrt{N}} \sum \vert {a}_{k} \vert exp ( j {ϕ}_{k} ) ,$$

where aₖ and φₖ are the amplitude and the phase of the k^{th} contribution respectively, N the number of diffusers in the medium. This amplitude appears as a random walk in the complex plane. In addition, the following hypotheses are considered:

(i) the amplitude aₖ and the phase φₖ of the k^{th} contribution are independent of each other and of any other contribution, and

(ii) the phases φₖ are uniformly distributed on [0; 2π].

On the basis of these hypotheses, Goodman (see document [22]) has developed, using the central limit theorem, the probability density function (equation (1)) for the real and imaginary parts of the electric field:

$$P ( {A}^{( r )} , {A}^{( i )} ) = \frac{1}{2 π {σ}^{2}} exp \{ - \frac{{[ {A}^{( r )} ]}^{2} + {[ {A}^{( i )} ]}^{2}}{2 {σ}^{2}} \} , \\ with {σ}^{2} = {lim}_{N -> ∞} \{ inf \frac{1}{N} {\sum }_{k = 1}^{N} \frac{〈 {\vert {a}_{k} \vert}^{2} 〉}{2} \} \qquad ( 1 )$$

The amplitude has a Gaussian circular distribution. The probability density of the intensity I may then be calculated and is expressed by:

$$P ( I ) = \frac{1}{2 {σ}^{2}} exp ( - \frac{I}{2 {σ}^{2}} ) \qquad ( 2 )$$

The intensity has a distribution of the decreasing exponential type. However, the intensity observed is that which is detected by the camera and thus corresponds to the space-time integration of this absolute intensity. Thus, the probability density function of the intensity detected I_{d} may be expressed as the convolution product of the absolute intensity and a detection function H:

I_{d}=∫∫I(u,v).H(x−u,y−v)dudv (3)

The probability density of the intensity detected is then expressed as:

$$P ( {I}_{d} ) = {( \frac{M}{〈 I 〉} )}^{M} \frac{{I}_{d}^{M - 1}}{Γ ( M )} exp ( - \frac{M \cdot {I}_{d}}{〈 I 〉} ) with M = {〈 I 〉}^{2} / {σ}_{I}^{2} \qquad ( 4 )$$

where σ_{I} is the standard deviation of the intensity, Γ(M) the usual gamma function:

$$Γ ( M ) = {\int }_{0}^{∞} {t}^{M - 1} exp ( - t ) d t$$

and M may be interpreted as the number of speckle grains seen by the camera. The intensity tends towards a Gaussian distribution when M tends towards +∞. Experimentally, a Gaussian distribution is observed for M much greater than 1. As a result, it is considered that the intensity detected follows a Gaussian process.

Second Order Statistic

Here we are interested in the representation of the experimental speckle in the frequency domain. Thus, we are no longer interested in its characteristic in one point of space (amplitude, intensity, phase) but between two points of space, in other words in what is known as its second order statistic.

The power spectral density (PSD) of a signal is defined as being the square of the module of the Fourier transform of this signal. The power spectral density of the intensity at one point having coordinates (x,y) is expressed as:

PSD(I(x,y))=|TF(I(x,y))|² (5)

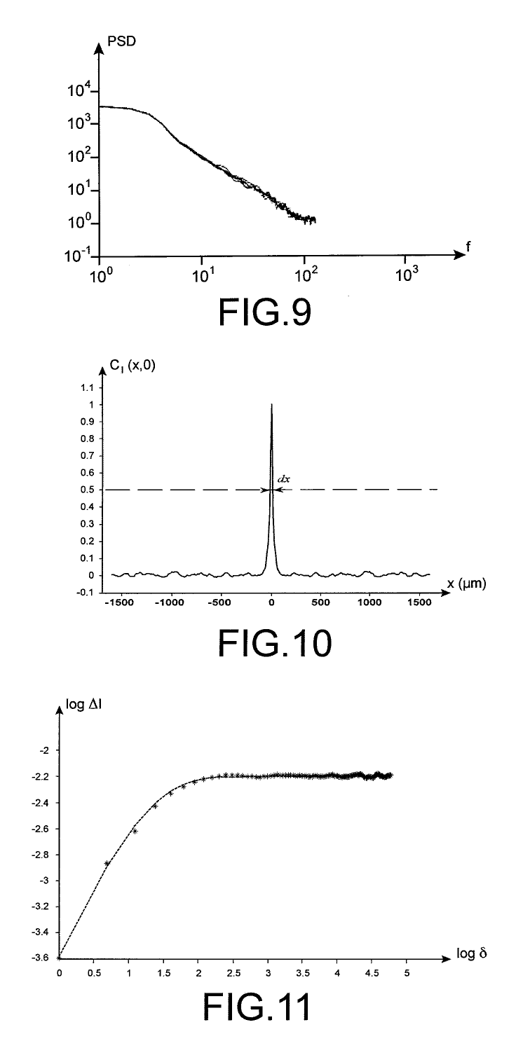

FIG. 9 shows a power spectral density PSD, which is typical of experimental speckle figures, as a function of the spatial frequency f, in log-log scale. It may be seen that the speckle figures show a decrease known as 1/f for high frequencies. This behaviour is characteristic of an autosimilar process in this frequency domain.

The spatial autocorrelation function in intensity is defined by equation (6):

R_{I}(Δx,Δy)= [symbol] I(x₁,y₁)I(x₂,y₂) [symbol] (6)

where Δx=x₁−x₂ and Δy=y₁−y₂. I(x₁,y₁) and I(x₂,y₂) are the intensities at two points of the observation plane (x,y). The symbol [symbol] [symbol] corresponds to the space average. If x₂=0, y₂=0, xᵢ=x and y₁=y, it is then possible to write:

R_{I}(Δx,Δy)=R_{I}(x,y).

The autocovariance function is defined as the autocorrelation function centred on the average. When it is normalised, it is expressed as:

$${c}_{I} ( x , y ) = \frac{{R}_{I} ( x , y ) - {〈 I ( x , y ) 〉}^{2}}{〈 {I ( x , y )}^{2} 〉 - {〈 I ( x , y ) 〉}^{2}} \qquad ( 7 )$$

According to the Wiener-Khintchine theorem, the autocorrelation function of the intensity is given by the inverse Fourier transform (designated FT⁻¹) of the PSD of the intensity:

R_{I}(x,y)=FT⁻¹[PSD(I(x,y))] (8)

This expression is used for the calculation of the autocorrelation function.

The calculated normalised autocovariance function is expressed as:

$${c}_{I} ( x , y ) = \frac{F {T}^{- 1} ( {\vert F T ( I ( x , y ) ) \vert}^{2} ) - {〈 I ( x , y ) 〉}^{2}}{〈 {I ( x , y )}^{2} 〉 - {〈 I ( x , y ) 〉}^{2}} \qquad ( 9 )$$

c_{I}(x,0) and c_{I}(0, y) correspond respectively to the horizontal and vertical profiles of c_{I}(x,y).

Their full widths at half maximum, respectively designated dx and dy, provide a reasonable measurement of the “average size” of the grains of a speckle figure (see document [20]).

FIG. 10 shows the horizontal profile c_{I}(x,0) as a function of x (in μm).

This constitutes the conventional frequential approach of the speckle phenomenon and then makes it possible to characterise spatially a speckle figure by what is known as “the speckle size”, by means of the characteristics of its grains.

Correlation Between the Speckle Phenomenon and Fractional Brownian Motion

Brownian motion is a mathematical description of the random motion sustained by a particle in suspension in a fluid, which is not subjected to any other interaction than that of the molecules of the fluid. The path of the particle in suspension is rendered random by the random fluctuations of the speeds of the molecules of the fluid. At the macroscopic scale, a random and disordered movement of the particle is observed.

If x={x(t), t ε [symbol] } designates the process characterising a Brownian motion phenomenon ( [symbol] : set of real numbers), the equation of its increments is expressed as:

[symbol]

[X(t+Δt)−X(t)]² [symbol] ∝Δt (10)

where the symbol ∝ signifies “proportional to”.

The correlation between the statistic of the speckle and that of Brownian motion has been proposed previously (see document [12]). Indeed, it should be recalled that in the speckle theory, the non-correlation between the amplitudes and the phases as well as between the increments (hypotheses (i) considered above) is assumed.

As a result, from the point of view of signal processing, the amplitude of the speckle corresponds to a Gaussian white noise. The Brownian motion is the integration of the Gaussian white noise. The intensity detected of the speckle then corresponds to a Brownian motion. Consequently, the first order statistics are of the same nature: they are Gaussian for the amplitude distribution and for the intensity distribution.

Their second order statistics also have the same characteristics: their PSD show a decrease in 1/f and their increments are Gaussian in both cases.

For this reason, the modelling of the speckle phenomenon by fractional Brownian motion has been considered (see document [12]). Equation (11) corresponds to the expression of the process of incrementing fractional Brownian motion. When the parameter H becomes equal to 0.5, this process becomes that of a conventional Brownian motion where there is no correlation between the increments (Eq. (10)).

[symbol]

[X(t+Δt)−X(t)]² [symbol] ∝Δt^{2H}, with Hε[0; 1] (11)

In fact, fractional Brownian motion is the generalisation of Brownian motion for which there is no correlation between the increments. Equation (11) is known under the name of diffusion equation.

In the present invention, the fractal approach of the speckle by the fractional Brownian motion model is applied to the study of the speckle coming in vivo from biological media.

Fractional Brownian motion applied to the speckle phenomenon: diffusion function of a speckle figure.

To describe the diffusion equation of a speckle figure, it is necessary to express the increment process for the intensity in the scale of spaces. With the second order stationarity hypothesis, one may write for the horizontal dimension of the image:

[symbol]

[I(x+Δx,y)−I(x,y)]² [symbol] =2( [symbol] I(x,y)² [symbol] −C_{ff}), (12)

where C_{ff} is the autocorrelation function of the intensity for the horizontal dimension of the image.

As has been seen previously, the PSD of the speckle contains a decrease in 1/f only for high frequencies. This behaviour for high frequencies characterises a local regularity on the trajectory of the increments. However, according to the fractal theory (see document [24]), the autocorrelation function of a process that contains a local regularity is:

C_{ff}= [symbol] X(t)X(t+Δt) [symbol] =σ²exp(−λ|Δt|^{2H}) (13)

where H reflects the Holderian regularity of the increments. The diffusion equation is then expressed, in the scale of spaces and for the horizontal dimension of the image, (see document [12]):

[symbol]

[I(x+Δx,y)−I(x,y)]² [symbol] =2σ_{I}²(1−exp(−λ|Δx|^{2H})) (14)

or:

log( [symbol] [I(x+Δx,y)−I(x,y)]² [symbol] )=log(2σ_{I}²)+log((1−exp(−λ|Δx|^{2H})) (15)

A graphical representation of equation (15) as well as the diffusion curve of a speckle figure obtained with sound skin are shown in FIG. 11 (arbitrary units). The dotted lines correspond to the theoretical curve and the stars to the experimental points. The increment of intensity is designated ΔI and the neighbourhood is designated δ.

Three parameters may be extracted from the diffusion curve, namely H, S and G:

H, the Hurst coefficient, is given by the slope at the origin. It is linked to the fractal dimension D_{f} of the image by the expression D_{f}=d+1−H, where d is the topological dimension. H characterises the fractal dimension of the image and is then a characteristic of the grains. It is also a local regularity parameter, as has been seen above.

S, the autosimilarity, is given by π/λ (see document [25]) and enables the quantification of the dimension in the image, the dimension that separates conventional behaviour from autosimilar behaviour. In this dimension, the process is said to have a “scale invariance”.

G, the saturation of the variance, equal to 2σ_{I}², characterises the image in an overall manner.

It should be noted that the linear part of the curve indicates the autosimilar behaviour of the process.

The documents cited in the present description are as follows:
[1] Benderitter M, Isoir M, Buand V, Durand V, Linard C, Vozenin-Brotons M C, Steffanazi J, Carsin H, Gourmelon P, “Collapse of skin antioxidant status during the subacute period of cutaneous radiation syndrome: a case report”, Radiat Res. 2007 January; 167(1):43-50
[2] Pouget J P, Laurent C, Delbos M, Benderitter M, Clairand I, Trompier F, Stephanazzi J, Carsin H, Lambert F, Voisin P, Gourmelon P, “PCC-FISH in skin fibroblasts for local dose assessment: biodosimetric analysis of a victim of the Georgian radiological accident”, Radiat Res. 2004 October; 162(4):365-76
[3] F. Daburon, “Irradiation aiguës localisëes, Bases radiobiologiques du diagnostic et du traitement”, EDP Sciences (1997).
[4] F Boulvert, B Boulbry, G Le Brun, B Le Jeune, S Rivet and J Cariou, “Analysis of the depolarizing properties of irradiated pig skin”, J. Opt. A: Pure Appl. Opt. vol 7, 21-28 (2005)
[5] J. D. Briers, G. Richards and X. W. He, “Capillary blood flow monitoring using laser speckle contrast analysis (LASCA)”, J. Biomed. Opt. 4, 164-175 (1999)
[6] Y. Aizu, T. Asakura, “Bio-speckle phenomena and their application to the evaluation of blood flow”, Opt. Las. Tech. 23, 205-219 (1991)
[7] I. V. Fedosov, V. V. Tuchin, “The use of dynamic speckle field space-time correlation function estimates for the direction and velocity determination of blood flow”, Proc. SPIE Vol. 4434, p. 192-196, Hybrid and Novel Imaging and New Optical Instrumentation for Biomedical Applications, Albert-Claude Boccara; Alexander A. Oraevsky; Eds.
[8] J. R. Tyrer, “Theory for three-dimensional measurements in ESPI”; Chapter 6 of Optical measurement methods in biomechanics, edited by J. F. Orr, London: Chapman & Hall (1997)
[9] J. F. Roman, P. Fernandez, V. Moreno, M. Abeleira, M. Gallas, D. Suarez, “The mechanical behavior of human mandibles studied by Electronic Speckle Pattern Interferometry”, The European Journal of Orthodontics, Vol. 21, No. 4, pp. 413-421 (1999)
[10] WO 2006/069443, Z. Haishan and L. Tchvialeva
[11] Y. Piederrière, J. Cariou, Y. Guern, B. Le Jeune, G. Le Brun, J. Lotrian, “Scattering through fluids: speckle size measurement and Monte Carlo simulations close to and into the multiple scattering”, Opt. Express 12, 176-188 (2004)
[12] S. Guyot, M. C. Péron, E. Deléchelle, “Spatial Speckle Characterization by Brownian Motion analysis”, Phys. Rev. E 70, 046618 (2004)
[13] US 2004/152989, J. Puttappa et al.
[14] L. Pothuaud, et al., “Fractal analysis of trabecular bone texture on radiographs: discriminant value in post menopausal osteoporosis”, Osteoporos. Int., Vol. 8, pp. 618-625 (1998)
[15] C. L. Benhamou, et al., “Fractal Analysis of radiographic Trabecular Bone Texture and Bone Mineral Density: Two Complementary Parameters Related to Osteoporotic Fractures”, Journal of bone and mineral research, Vol. 16, No. 4, pp. 697-704 (2001)
[16] L. Zhifand, L. Hui and Y. Qiu, “Fractal analysis of laser speckle for measuring roughness”, Proc. SPIE., Vol. 6027, pp. 470-476 (2006)
[17] O. Carvalho et al., “Statistical speckle study to characterize scattering media: use of two complementary approaches”, Optics Express, Vol. 15, No 21, pp. 13817-13931 (2007)
[18] O. Carvalho et al., “Speckle: Tool for diagnosis assistance”, Proc. SPIE Int. Soc. Opt. Eng., Speckle 06: Speckles, from grains to flowers 2006, Vol. 6341, pp. 1-6 (2006)
[19] D. A. Boas and A. G. Yodh, “Spatially varying dynamical properties of turbid media probed with diffusing temporal light correlation”, J. Opt. Soc. Am. A, Vol. 14, No. 1, 192-215 (1997)
[20] Q. B. Li and F. P. Chiang, “Three-dimensional dimension of laser speckle”, Applied Optics, Vol. 31, No. 29, 6287-6291 (1992)
[21] R. V. Hogg, and J. Ledolter, J. Engineering statistics, Macmillan Publishing Company (1987)
[22] J. W. Goodman, “Statistical Properties of Laser Speckle Patterns”, in Laser speckle and related phenomena, Vol. 9 in the series Topics in Applied Physics, J. C. Dainty, Ed., (Springer-Verlag, Berlin, Heidelberg New York Tokyo, 1984).
[23] L. I. Goldfisher, “Autocorrelation function and power spectral density of last-produced speckle pattern”, J. Opt. Soc. Am., Vol. 55, No. 3, 247-253 (1964)
[24] P. Abry, P. Gonçalves, P. Flandrin, Spectrum analysis and 1/f processes, Springer, Berlin (1995)
[25] T. D. Frank, A. Daffertshofer, P. J. Beek, “Multivariate Ornstein-Uhlenberg processes with mean field-dependent coefficients-application to postural sway”, Phys. Rev., Vol. 63 (2001)

1. Device for measuring in vivo properties of biological tissues, in particular for the diagnostic and prognostic assistance of physiopathological changes, particularly tissular lesions and more particularly by irradiation, for the evaluation of cutaneous ageing, for the evaluation of the efficacy of cosmetological or dermatological products, said device being characterised in that it comprises:
a coherent light source (13) for emitting a coherent light along a first direction (X), for the purpose of illuminating a biological tissue (16) in first and second areas thereof, the first area being sound and the second area liable to include changes, the tissue thus illuminated generating a speckle phenomenon,
observation and acquisition means (14) for observing the speckle field in a second direction (Y) and for acquiring the speckle,
means (18) for varying the angle between the first and second directions, in order to observe the speckle field at different angles, so as to acquire information about the tissue at various depths in this tissue,
support and absorption means (20, 28) to maintain a constant distance between the point of illumination of the surface of the tissue and the observation and acquisition means and to absorb possible movements of the tissue,
electronic means (22) for processing the speckle figures obtained by means of the observation and acquisition means, in order to compare the first and second areas, and
electronic means (24) for analysing the processing of the figures, by statistical methods, making it possible to validate the comparison made between the first and second areas.

2. Device according to claim 1, wherein the means for varying the angle between the first and second directions are capable of varying this angle in the interval ranging substantially from 0° to 180°.

3. Device according to claim 1, wherein the means for varying the angle between the first and second directions are capable of modifying the orientation of the first direction (X) independently of that of the second direction (Y) and inversely.

4. Device according to claim 1, further comprising optical means that are capable of controlling the polarisation of the coherent light emitted by the source and the polarisation of the light arriving on the observation and acquisition means.

5. Device according to claim 1, wherein
the observation and acquisition means comprise photodetection means (14) that capture the speckle and supply electrical signals representative of the corresponding speckle figures, and
the electronic means for processing (22) are capable of processing the electrical signals in the form of non-compressed images, and of making it possible to compare the first and second areas.

6. Device according to claim 5, wherein the photodetection means (14) are capable of capturing the speckle with exposure times of at most 100 μs.

7. Device according to claim 5, wherein the photodetection means comprise a camera (14).

8. Device according to claim 7, wherein the camera (14) is a CCD camera.

9. Device according to claim 1, wherein the observation and acquisition means (14) are provided to acquire at least 200 speckle figures per illuminated area.

10. Device according to claim 1, wherein the coherent light source (13) is monochromatic.

11. Device according to claim 1, wherein the processing of the speckle figures is carried out by a fractal method or by a conventional frequential method or both.

12. Device according to claim 11, wherein the processing of the speckle figures, when it is carried out by a fractal method, comprises the extraction of stochastic parameters that are characteristic of the speckle figures.

13. Device according to claim 12, wherein the stochastic parameters comprise:
the Hurst coefficient,
the autosimilarity, and
the saturation of the variance.

14. Device according to claim 2, wherein the means for varying the angle between the first and second directions are capable of modifying the orientation of the first direction (X) independently of that of the second direction (Y) and inversely.

15. Device according to claim 6, wherein the photodetection means comprise a camera (14).

16. A method for measuring in vivo properties of biological tissues, in particular for the diagnostic and prognostic assistance of physiopathological changes, particularly tissular lesions and more particularly by irradiation, for the evaluation of cutaneous ageing, for the evaluation of the efficacy of cosmetological or dermatological products, said method comprising:
emitting a coherent light along a first direction, for the purpose of illuminating a biological tissue in first and second areas thereof, the first area being sound and the second area liable to include changes, the tissue thus illuminated generating a speckle phenomenon,
observing and acquiring the speckle in a second direction,
for varying the angle between the first and second directions, in order to observe the speckle field at different angles, so as to acquire information about the tissue at various depths in this tissue,
absorbing possible movements of the tissue and maintaining a constant distance between the point of illumination of the surface of the tissue and means for observing and acquiring the speckle,
processing the speckle figures obtained in the first and second areas, in order to compare the first and second areas, and
analysing the processing of the figures, by statistical methods, making it possible to validate the comparison made between the first and second areas.

17. The method according to claim 16, wherein the angle between the first and second directions is varied in the interval ranging substantially from 0° to 180°.

18. The method according to claim 16, wherein the angle between the first and second directions is varied by modifying the orientation of the first direction independently of that of the second direction and inversely.

19. The method according to claim 16, further comprising controlling the polarisation of the coherent light emitted towards the biological tissue and the polarisation of the observed and acquires light.

20. The method according to claim 16, wherein
the speckle figures are acquired in such a way that they are non-compressed.

21. The method according to claim 16, wherein the speckle is captured with exposure times of at most 100 μs.

22. The method according to claim 16, wherein at least 200 speckle figures are acquired per illuminated area.

23. The method according to claim 16, wherein the processing of the speckle figures is carried out by a fractal method or by a conventional frequential method or both.

24. The method according to claim 23, wherein the processing of the speckle figures, when it is carried out by a fractal method, comprises the extraction of stochastic parameters that are characteristic of the speckle figures.

25. Device according to claim 24, wherein the stochastic parameters comprise:
the Hurst coefficient,
the autosimilarity, and
the saturation of the variance.
